(19) United States
(12) Patent Application Publication
(10) Pub. No.: US 2008/0070267 A1
(43) Pub. Date: Mar. 20, 2008

(54) Modulation Of Neurotransmitter Activity In Neurons

(75) Inventors: Nicholas C. Spitzer, Del Mar, CA (US); Laura Borodinsky, Sacramento, CA (US); Cory M. Root, San Diego, CA (US)

Correspondence Address: BUCHANAN, INGERSOLL & ROONEY LLP, P.O. BOX 1404, ALEXANDRIA, VA, 22313-1404, US

(73) Assignee: The Regents of the University of California, Oakland, CA (US)

(21) Appl. No.: 11/596,638

(22) PCT Filed: May 13, 2005
(86) PCT No.: PCT/US05/16851

§ 371 Date: Nov. 13, 2006

Related U.S. Application Data
(60) Provisional application No. 60/573,683, filed on May 20, 2004.

(51) Int. Cl. C12Q 1/02; A61P 25/00; C12N 13/00; C12N 5/00; C12N 5/08

(52) U.S. Cl. 435/29; 435/173.4; 435/368; 435/375

(57) ABSTRACT

The present invention relates generally to methods, devices and compositions for treating mental, neurological, and cognitive diseases related to deficiencies in the biosynthesis and/or metabolism of neurotransmitters.

### RELATED APPLICATIONS

This application claims the benefit of priority of provisional application Ser. No. 60/573,683, filed May 20, 2004, which is incorporated herein by reference.

### TECHNICAL FIELD

The present invention relates generally to methods, devices, and compositions for treating mental, neurological, and cognitive diseases related to deficiencies in the biosynthesis and/or metabolism of neurotransmitters.

### BACKGROUND

Neurotransmitters are essential for interneuronal signaling, and the specification of appropriate transmitters in differentiating neurons has been related to intrinsic neuronal identity and to extrinsic signaling proteins. The determination of neuronal phenotypes is a substantial developmental challenge, given the complexity of the nervous system. The classical low-molecular-mass, peptide, gaseous, and growth factor neurotransmitters number 50 or more.¹ The appearance of a particular transmitter in a given class of neurons is a crucial step in differentiation because it enables the neurons to communicate with others with which they make synaptic connections. Expression of an incorrect transmitter could isolate neurons from their normal networks. The absence of synaptic signaling could also reduce trophic support from its postsynaptic partners,² leading to neuronal death.

Several classes of mechanisms regulate transmitter expression. Specification by intrinsic transcription factors has been demonstrated by the ectopic expression of MNR2 or Lhx3/Isl1 in the chick spinal cord, which drives inappropriate expression of the motor neuron transmitter acetylcholine in interneurons.^{3,4} In mice that are mutant for the DBx1 transcription factor, neural progenitors from the Dbx1 domain give rise to interneurons with an inappropriate GABAergic phenotype.⁵ Gain-of-function and loss-of-function experiments with homeodomain transcription factors in Caenorhabditis elegans and Drosophila led to the misexpression of GABA and a loss of synthesis of dopamine and serotonin.^{6,7} Cytokines and neurotrophic factors can also regulate transmitter expression and can drive the expression of acetylcholine instead of noradrenaline (norepinephrine) in rat sympathetic ganglion neurons, both in culture and in vivo.⁸⁻¹⁰ Additionally, the imposition of activity can regulate the choice of neurotransmitter in cultured neurons by means of Ca²⁺ influx¹¹ and can differentially affect the regulation of transmitter expression by protein factors.¹² The incidence of neurons expressing the transmitter GABA and its synthetic enzyme, glutamic acid decarboxylase (GAD), is up-regulated in cultured embryonic spinal neurons by increasing the frequencies of Ca²⁺ spikes that mimic endogenous spontaneous activity.^{13,14}

Many illnesses and disorders result from the over- or under-production of neurotransmitters. Examples of such illnesses and disorders include psychiatric illnesses such as schizophrenia, manic-depression, obsessive-compulsive disorder, and addiction. Treatment of these disorders is presently available. The primary existing treatment for manic-depressive illness, for example, is pharmacological, involving the use of drugs that affect the metabolism of neurotransmitters. Some drugs block the uptake of transmitters, thus increasing the amount of a transmitter available to bind to neurotransmitter receptors; other drugs deplete the stores of transmitters in the neurons, decreasing the stores of transmitters that the neurons have available to release. These drugs are relatively selective, but have unwanted side effects. A secondary existing treatment for manic-depressive illness, for example, is electroconvulsive (shock) therapy (ECT). This entails generalized stimulation of the nervous system to produce a seizure, and is performed while the patient is anesthetized. Like the current pharmacological treatment of neurological and psychological disorders, ECT is not a focused treatment and has unwanted side-effects. Furthermore, ECT is used principally to treat the most severe cases of cognitive dysfunction that are refractory to pharmacological therapy. Thus, there is a need for a more selective therapy with fewer side effects for treatment of psychological and neurological disorders.

### SUMMARY

This application provides, among others, a method for modulating the neurotransmitter activity of neurons, allowing for the treatment of various psychological and neurological disorders and permitting the screening of potential candidate neuromodulators useful in the treatment of various psychological and neurological disorders and illnesses. In one embodiment, a method of modulating neurotransmitter activity in a neuron associated with the central nervous system is provided. The method includes contacting the neuron with a stimulatory factor that alters the pattern of Ca²⁺ spike activity of the neuron. The neuron can be a fully differentiated adult neuron or embryonic neuron. The stimulatory factor can be electrical or chemical. The neurotransmitter can be acetylcholine, nitric oxide, histamine, noradrenaline, a bioactive amine, an amino acid or a neuropeptide. Generally, the modulation of neurotransmitter activity comprises altering neurotransmitter expression.

In another embodiment, a method of treating or inhibiting a psychological disorder in a subject is provided. In one embodiment, the method includes administering to the subject a stimulatory or inhibitory factor that alters the pattern of Ca²⁺ spike activity of neurons, thereby resulting in the modification of neurotransmitter activity produced by the neurons. In one embodiment, the psychological disorder is selected from the group consisting of addiction, substance abuse, autism, dyslexia, obsessive-compulsive disorder, generalized anxiety disorder, post-traumatic stress disorder, panic attacks, social phobia, major depression, bipolar disorder and schizophrenia.

In another embodiment, a method of altering neurotransmitter expression is provided. In one embodiment, the method includes contacting a neuron comprising a nucleic acid sequence encoding a neurotransmitter, or a nucleic acid sequence encoding an enzyme necessary for the biosynthesis of the neurotransmitter, with a stimulatory factor that alters the pattern of Ca²⁺ spike activity of the neuron.

In another embodiment, a method of screening neuromodulators that could alter the frequency of calcium spikes is provided. In one embodiment, the method includes using cultures of neurons prepared from developing embryos and loading the cultured cells with a calcium indicator such as fluo-4AM. In certain embodiments, after neurons are exposed to different neuromodulators, time lapse imaging is used to assay changes in the firing pattern of calcium spikes. In another embodiment, promising types or concentrations of neuromodulators can then be tested in an in vitro assay by imaging neurons in the intact spinal cord in partially dissected embryos, allowing identification of the classes of neurons that are affected by these neuromodulators and also allowing the exclusion of any artifacts of cell culture.

### DESCRIPTION OF THE DRAWINGS

FIG. 1 depicts Ca²⁺ spike activity of four classes of neurons imaged in the embryonic spinal cord. a, Active Rohon-Beard neurons (RB, continuous circles) and dorsolateral interneurons (DLI, dashed circles) on the dorsal surface of a stage 23 neural tube; insets illustrate spike activity for cells indicated by arrows during a 1-h period; F/F₀, fluorescence increase above baseline. b, Whole-mount immunoreactivity for HNK-1 identifies RBs on the dorsal surface of the same preparation. c, Coactive motor neurons (MN, continuous circles) and ventral interneurons (VI, dashed circles) on the ventral surface of a stage 24 neural tube; insets illustrate spike activity for cells indicated by arrows during a period of 1 h; scale as in a. d, Whole-mount immunoreactivity for lim-3 identifies MNs on the ventral surface of the same preparation. e, Incidence of Ca²⁺ spiking (percentage active cells) for these neurons during three developmental periods. f, Frequency of Ca²⁺ spikes (spikes h⁻¹) for these neurons, excluding neurons that were silent during the imaging period.

FIG. 2 depicts suppression of spike activity in vivo by overexpression of inward rectifier K⁺ channels and the subsequent increase in the incidence of expression of glutamatergic and cholinergic phenotypes. a, Experimental design. b, Neural tube resulting from unilateral injection of transcripts plus tracer (left), loaded with bisoxonol (BISOX) to image membrane potential right). c, Neural tube resulting from unilateral injection of transcripts plus tracer left), loaded with fluo-4 acetoxymethyl ester to image spikes (middle). d, Neural-tube sections from control embryos stained for glutamate (Glu) or the vesicular glutamate transporter (VGluT) in combination with HNK-1, and choline acetyltransferase (ChAT) in combination with lim-3. e, Embryos unilaterally silenced; f, Bilaterally silenced and stained as in d.

FIG. 3 depicts enhancement of spike activity in vivo by overexpression of voltage-gated Na⁺ channels and the subsequent decrease in the incidence of glutamatergic and cholinergic phenotypes. a, Neural tube after unilateral injection of transcripts plus tracer (left) and fluo-4 imaging (right) shows that spike frequency is enhanced in dorsal neurons containing transcripts; spiking cells are circled. b, Spike incidence in dorsal and ventral neurons after unilateral or bilateral injection of transcripts and tracer. c, Spike frequency in RB, DLI, MN and VI marked with tracer. d, e, Embryos with unilateral and bilateral enhancement of activity, stained for glutamate or VGluT in combination with HNK-1 or for ChAT in combination with lim-3.

FIG. 4 depicts the pharmacological in vivo suppression of spikes with Ca²⁺ and Na⁺ channel blockers or enhancement of spikes with the sodium channel agonist veratridine enhances or suppresses Glu-IR and ChAT-IR, respectively. a, Experimental design. b, Incidence of spike activity in dorsal and ventral neurons in the presence of Ca²⁺ and Na⁺ channel blockers or veratridine. c, Spike frequency in RB, DLI, MN and VI in the presence of veratridine. d, Bead implanted in a stage 18 embryo sectioned at stage 40. e, f, Glu/HNK-1 staining and ChAT/lim 3 staining following implantation of beads containing blockers (e) or veratridine (f).

FIG. 5 depicts suppression or enhancement of spike activity in vivo and the subsequent homeostatic superposition or replacement of one transmitter with another. a, Controls doubly stained for glutamate or ChAT (dark gray, excitatory) plus glycine or GABA (light gray, inhibitory). b, Embryos in which spike activity was bilaterally suppressed by expression of hKir2.1, stained as in a. c, Embryos in which spike activity was bilaterally enhanced by expression of rNaᵥ2a, stained for glutamate or ChAT and the respective marker of cell identity (HNK-1 or lim-3, white), plus glycine or GABA.

FIG. 6 depicts regulation of spike frequency in vitro driving novel expression of neurotransmitters. a, Examples of Glu⁺/HNK-1⁺ and Glu⁺/HNK-1 phenotypes, and Glu-IR and HNK-1-IR in 2 mM Ca²⁺ (white) or after suppression of spike activity with 0 mM Ca²⁺ (solid gray) or 2 mM Ca²⁺ culture medium with bis-(o-aminophenoxy)ethane-N,N,N,N-tetraacetic acid acetoxymethyl ester (BAPTA-AM, dotted), Ca²⁺ channel blockers (horizontal hatching) or hKir2.1 expression (vertical hatching). b, Examples of ChAT⁺/lim-3⁺ and ChAT⁺/lim-3 phenotypes, and ChAT-IR and lim-3-IR in 2 mM Ca²⁺ (white) or after suppression of spike activity as in a. c, Staining for Glu (circles), VGluT (triangles) and HNK-1 (squares) as a function of frequency of imposed Ca²⁺ spikes. d, Staining of ChAT (circles) and lim-3 (squares) as a function of frequency of imposed Ca²⁺ spikes. e, Proportions of Glu⁺/ChAT⁺ doubly stained neurons when cultured in 2 mM Ca²⁺ or 0 mM Ca²⁺ medium (indicated by 2 or 0, respectively). f, Neurons doubly labeled for excitatory (light gray) and inhibitory (dark gray) transmitters; lighter gray indicates coexpression. g, Incidence of neurons expressing excitatory and inhibitory transmitters when grown in 2 mM Ca²⁺ or 0 mM Ca²⁺ culture medium (indicated by 2 or 0, respectively).

FIG. 7 depicts the functional release of neurotransmitters expressed after alterations in Ca²⁺ spike activity. a, Whole-cell recording from a myoball expressing native AChR manipulated in front of the growth cone of a neuron grown in 2 mM Ca²⁺ culture medium. b, Similar recording from a myoball expressing rGluR2 in front of another growth cone of a neuron grown in 0 mM Ca²⁺ medium, in the absence and presence of CNQX. c, Percentage of neurons generating cholinergic (ACh) or glutamatergic (Glu) SSCs when cultured in 2 mM Ca²⁺ or 0 mM Ca²⁺ medium (indicated by 2 or 0, respectively) and tested with control myoballs or myoballs expressing GluR2; all recordings were made in the presence of 2 mM Ca²⁺ (n>9 for each condition).

FIG. 8 depicts critical periods for Ca²⁺ spike-dependent regulation of transmitter expression in vitro. a, Experimental design. b, Glutamate staining as a function of the period of initial stimulation (circles, spike frequency 10 h⁻¹) or deprivation (triangles, spike frequency 0 h⁻¹), and ChAT staining as a function of the period of initial stimulation or deprivation.

FIG. 9 depicts calcium-dependent expression of GABA and GAD.

FIG. 10 depicts whole mount in situ hybridization of xGAD 67.

FIG. 11 depicts a comparison of spontaneous calcium spikes and stimulated calcium spikes.

FIG. 12 depicts the relationship between the frequency of calcium spikes and xGAD 67 transcript expression.

FIG. 13 depicts a model for neurotransmitter specification based on studies of the Xenopus spinal cord.

### DETAILED DESCRIPTION

We have examined the role of electrical activity and Ca²⁺ influx in the specification of neurotransmitter expression in the developing spinal cord of Xenopus laevis embryos. This is an attractive system to study because it contains only eight classes of neurons that collectively express four classical transmitters.^{15,16} Imaging neurons of four of these classes, we find they generate distinct patterns of Ca²⁺ spikes in vivo shortly after neural tube formation, starting before the synapse formation that enables network activity.¹⁷ We show that suppressing or enhancing this activity in the neural tube, either by misexpression of K⁺ or Na⁺ channels or with pharmacological antagonists and agonists, alters the expression of acetylcholine and glutamate without affecting markers of neuronal identity. In a homeostatic manner, spike suppression increases the incidence of excitatory transmitter expression and decreases the incidence of inhibitory transmitters. Conversely, enhancing spike production decreases the expression of excitatory transmitters and increases the expression of inhibitory transmitters. Furthermore, we show the activity-dependent homeostatic specification of neurotransmitter receptors, indicating that changing the transmitter in a population of neurons is likely to be functional. We also show that by altering electrical activity, we can alter the number of neurons expressing specific neurotransmitters, such as serotonin and dopamine, in the brain.^{18,19} Cell cultures permit the imposition of specific spike frequencies, and we find that transmitter expression in vitro is dependent on Ca²⁺ spike frequency. We demonstrate transmitter release from neurons that is inappropriate for the markers they express, indicating that transmitter switches are functional. Finally we show that the effects of activity on transmitter specification are restricted to a critical period at this early stage of development. We show that early expression of multiple neurotransmitters is pruned to single transmitters by electrical activity, and that the early expression of transmitters regulates this electrical activity.

Electrical and/or chemical neuromodulating techniques are provided. Accordingly, in one embodiment, the present invention relates to modulation of neuronal activity to affect neurological, psychological, or psychiatric activity. In one embodiment, the present invention finds application in the modulation of neuronal function or processing to affect a functional outcome. The modulation of neuronal function is useful with regard to the prevention, treatment, or amelioration of neurological, psychiatric, psychological, conscious state, behavioral, mood, and thought activity. (unless otherwise indicated these will be collectively referred to herein as “psychological activity” or “psychiatric activity”). When referring to a pathological or undesirable conditions associated with the activity, reference may be made to “psychiatric disorder” or “psychological disorder” instead of psychiatric or psychological activity. Although the activity to be modulated usually manifests itself in the form of a disorder such as addiction, substance abuse, autism, dyslexia, obsessive-compulsive disorder, generalized anxiety disorder, post-traumatic stress disorder, panic attacks, social phobia, major depression, bipolar disorder, or schizophrenia, it is to be appreciated that the invention may also find application in conjunction with enhancing or diminishing any neurological or psychiatric function, not just an abnormality or disorder. Psychiatric activity that may be modulated can include, but not be limited to, normal functions such as alertness, conscious state, drive, fear, anger, anxiety, euphoria, sadness, and the “fight or flight” response. Neurological disorders that may be modulated can include movement disorders such as Parkinson's disease, tardive dyskinesia, and Huntington's disease.

In one embodiment, the present invention provides methods and compositions for manipulating the electrical activity of the nervous system. The disclosed methods and compositions can be used to modify the identity of the neurotransmitter molecules that nerve cells synthesize and use to communicate with other nerve cells in the central nervous system. For example, the use of Transcranial Magnetic Stimulation (TMS) for modulating the activity and/or synthesis of neurotransmitters is encompassed by the invention. TMS applied at frequencies mimicking natural patterns of activity occurring in the brain; for example, frequencies on the order of ten magnetic pulses per hour can be used. Understanding the ways in which neurons express particular transmitters could have a profound impact on the way we think about treating mental illness, many forms of which result from disorders of neurotransmitter metabolism.

In one embodiment, the present invention provides a more selective and specific therapy for manic-depressive illness, schizophrenia, and perhaps other neurological or cognitive disorders than the pharmacological therapy and the gross electrical stimulation (e.g., electroconvulsive) therapy that presently exist.

In one embodiment, the present invention relates generally to modulating the pathological electrical and chemical activity of the brain by electrical stimulation and/or direct placement of neuromodulating chemicals within the central nervous system (CNS). In one embodiment, the invention provides for the treatment of, for example, psychiatric disorders (e.g. addictions, substance abuse, obsessive-compulsive disorder, generalized anxiety disorder, post-traumatic stress disorder, panic attacks, social phobia, major depression, bipolar disorder, and schizophrenia). Accordingly, in one embodiment, the invention includes an approach to treat mental and related cognitive diseases and movement and related neurological disorders that arise from deficiencies in the biosynthesis/metabolism of certain important neurotransmitters. The approaches can be medical devices or pharmaceuticals.

In one embodiment, the invention is to apply electrophysiology to alter the “electrical activity” of the brain or of the regenerating spinal cord. It has been discovered herein that, at least in an immature animal nervous system, when one changes the electrical activities of the system (monitored by measuring the Ca²⁺ spikes), the system changes the identities of the neurotransmitters it synthesizes. Therefore, for any disease that is deficient in the synthesis on a specific neurotransmitter, by manipulating its electrical activity, a different set of neurotransmitters may be synthesized to compensate.

The electrical stimulation of different regions of the young or adult brain, or regenerating spinal cord, will change the neurotransmitters that neurons synthesize and use to communicate with other neurons. This could be a powerful treatment for mental disorders, such as depression and schizophrenia, or neurological disorders, such as Parkinson's disease, tardive dyskinesia, and Huntington's disease, that are known to result from disorders of neurotransmitter metabolism. Such treatment would be focused and would avoid the side-effects caused by the existing, more generalized, therapies.

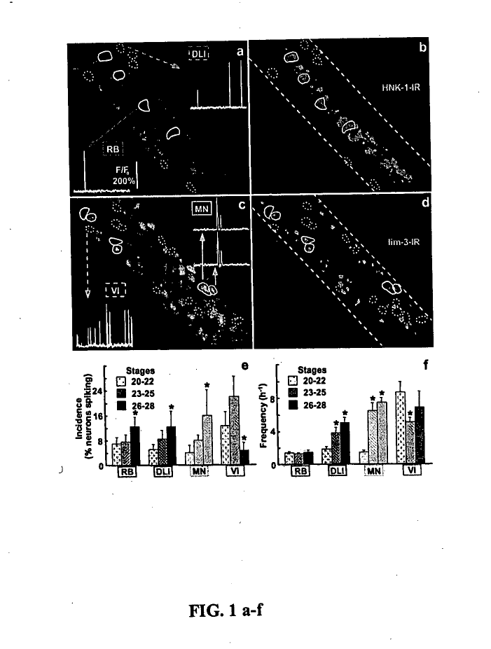

To determine patterns of neuronal activity in the embryonic spinal cord, we imaged spontaneous Ca²⁺ spikes²⁰ in dorsal sensory Rohon-Beard neurons (RB), dorsolateral interneurons (DLI), ventral motoneurons (MN) and ventral interneurons (VI) (FIG. 1a, c). We classified neurons on the dorsal surface of the neural tube dorsomedial and dorsolateral neurons by their positions. Neurons located along the midline of the dorsal spinal cord are sensory neurons,^{15,16} whole-mount immunocytochemistry with antibodies against HNK-1-a membrane glycoprotein and specifically expressed on RB cell bodies and processes²⁰-confirmed this identity (FIG. 1b). Note that in FIG. 1b, the dashed white lines indicate the margins of the neural tube. We classified neurons on the ventral surface of the neural tube on the basis of the presence of coactivity. Ventral neurons that fired in concert were considered to be MN,^{15,17} whereas neurons that did not spike together with others were designated VI. The clusters of MN identified by Ca²⁺ spike co-activity (FIG. 1c) co-localized with neurons immunostained with lim-3 transcription factor in the ventral neural tube (FIG. 1d). Note that in FIG. 1d, profiles of nuclei are of different sizes as the result of the through-series projection of a small stack of images and differences in nuclear orientation. All lim-3-immunoreactive neurons stained for choline acetyltransferase (ChAT), a generic MN marker,²² and vice versa (see below). Zebrafish VeLD interneurons express lim-3 and are GABA-immunoreactive,^{23,24} but we observe no GABA immunoreactivity in lim-3⁺ neurons. These results indicate further that at the Xenopus developmental stages evaluated, lim-3 is expressed only in MN.²⁵ During a 10-h developmental period after closure of the neural tube, the incidence of spike activity increases for RB, DLI and MN but decreases for VI. The frequency patterns of spikes are different for RB (low and constant), DLI (monotonically increasing), MN (step from low to high) and VI (high throughout) (FIG. 1e, f). In FIGS. 1e and f, n>10 embryos were used for each period. The neural tube is about 100 μm in diameter at these stages. In the figures, asterisks indicate values that are significantly different from stages 20-22. Dotted columns, stages 20-22; hatched columns, stages 23-25; solid columns, stages 26-28. These observations indicated that neurons might be recognizable by signatures of activity as well as by their position and expression of molecular markers, and raised the possibility that activity drives neurotransmitter phenotype. This notion led to the hypothesis that perturbing these patterns of activity would alter the specification of neurotransmitters.

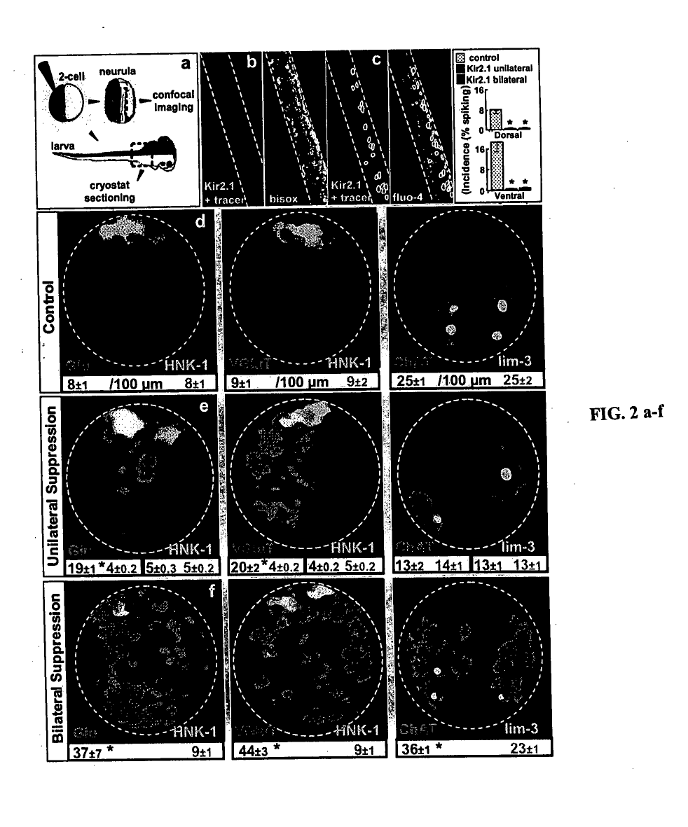

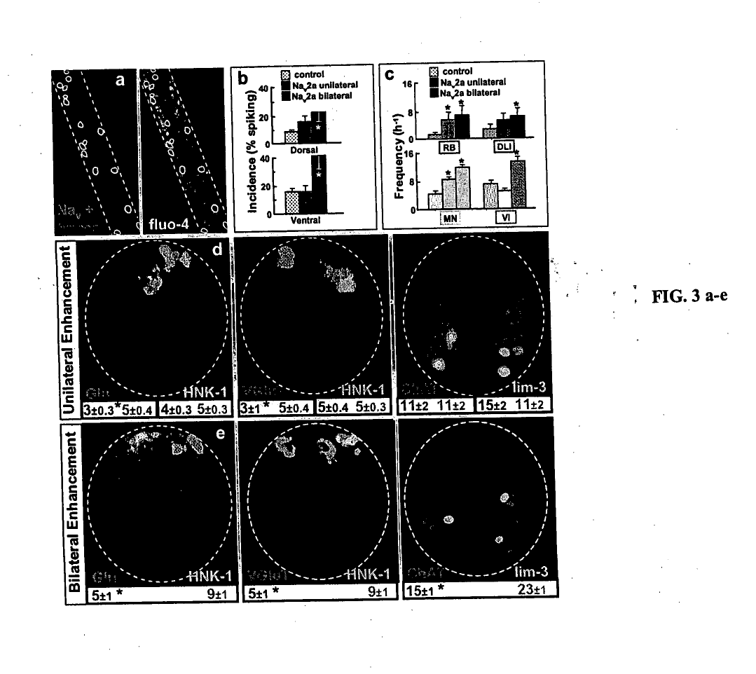

To examine the role of activity in neuronal differentiation, we suppressed Ca²⁺ spikes by overexpression of human inward rectifier K⁺ channels (hKir2.1) and later assessed the presence of neurotransmitters immunocytochemically (FIG. 2a). hKir2.1 transcripts and fluorescent tracer were injected together into one or both blastomeres at the two-cell stage. Ca²⁺ imaging was performed on stage 22-26 neural-tube embryos (boxed region in FIG. 2a) and stage 40 larvae were sectioned for immunocytochemistry. Imaging embryonic neural tubes loaded with the voltage-sensitive indicator bisoxonol revealed that unilateral expression of hKir2.1 causes the hyperpolarization of neurons only on the ipsilateral side. Imaging with fluo-4 demonstrated ipsilateral suppression of spontaneous spikes (FIG. 2b, c). FIG. 2b depicts the neural tube resulting from unilateral injection of transcripts plus tracer (left), loaded with bisoxonol (BISOX) to image membrane potential (right). FIG. 2b reveals that dorsolateral neurons containing transcripts are hyperpolarized (cells are pseudocolored blue, appearing dark gray or black in this figure; n=7 neural tubes). In FIG. 2b, white dashed lines indicate margins of the neural tube. FIG. 2c depicts the neural tube resulting from unilateral injection of transcripts plus tracer left), loaded with fluo-4 acetoxymethyl ester to image spikes (middle). FIG. 2c reveals that spikes in dorsal neurons are suppressed on the side containing transcripts (active cells are circled). In FIG. 2c, the incidence of spiking is reduced in both dorsal and ventral neurons marked with tracer (n=15 neural tubes). In FIG. 2c, dotted columns represent controls; hatched columns, Kir2.1 unilateral; solid columns, Kir2.1 bilateral. Bilateral expression hyperpolarized neurons and silenced spikes on both sides of the neural tube. Glutamate immunoreactivity (Glu-IR) and glutamate vesicular transporter immunoreactivity (VGluT-IR), and choline acetyltransferase immunoreactivity (ChAT-IR), are normally observed only in RB and MN, identified by HNK-1-IR and lim-3-IR, respectively (FIG. 2d). Strikingly, Glu-IR and VGluT-IR were present in HNK-1 cells expressing hKir2.1 either unilaterally or bilaterally (FIG. 2e, f). In FIGS. 2d-f, white dashed ovals indicate neural-tube perimeters. Numbers of immunoreactive neurons per 100 μm of neural tube are indicated beneath panels (means ±s.e.m., n>5 embryos). For unilaterally silenced embryos these numbers are tabulated separately for each side of the neural tube. As above, asterisks indicate significantly different from control. In contrast, ChAT-IR was present in lim-3 cells only after bilateral (and not unilateral) injections. Unilateral suppression might allow commissural axon projections from the contralateral side¹⁶ to prevent expansion of the cholinergic phenotype. The numbers of HNK-1⁺ and lim-3⁺ cells were not affected by either unilateral or bilateral suppression of activity, and the cellular organization of the neural tube seemed normal. These results indicate that suppression of activity might lead to the spread of glutamatergic and cholinergic phenotypes. To test whether increases in Ca²⁺ spike frequency suppress the incidence of Glu-IR and ChAT-IR, we overexpressed voltage-gated rat brain Na⁺ channels (rNaᵥ2aα and rNaᵥ2aβ). rNaᵥ2aαβ transcripts and fluorescent tracer were injected together into one or both blastomeres at the two-cell stage, followed by imaging and immunocytochemistry as in FIG. 2. Both the incidence of spiking neurons and spike frequency were increased as a result (FIG. 3a-c). FIG. 3a shows a neural tube after unilateral injection of transcripts plus tracer (left) and fluo-4 imaging (right), demonstrating that spike frequency is enhanced in dorsal neurons containing transcripts. In FIG. 3a, spiking cells are circled. In FIGS. 3b and c, n≧10 neural tubes were analyzed; dotted columns represent controls; horizontally hatched columns, Naᵥ2a unilateral; solid columns, Naᵥ2a bilateral. Glu-IR and VGluT-IR were decreased after overexpression of rNaᵥ2aαβ both unilaterally and bilaterally, and ChAT-IR was decreased in cells after bilateral increases in spike activity (FIG. 3d, e). In FIGS. 3d and e, for each condition, n≧5. For unilaterally silenced embryos these numbers are tabulated separately for each side of the neural tube. As above, asterisks indicate significantly different from control in FIGS. 3b-e. Unilateral expression of Na⁺ channels caused a decrease in activity in contralateral, unlabelled VI that might result from commissural axon projections. Consistent with this level of activity there was a significant increase in ChAT-IR in lim-3 cells on the contralateral side. In summary, suppression and enhancement of activity thus seem to exert opposing effects on expression of these excitatory transmitters.

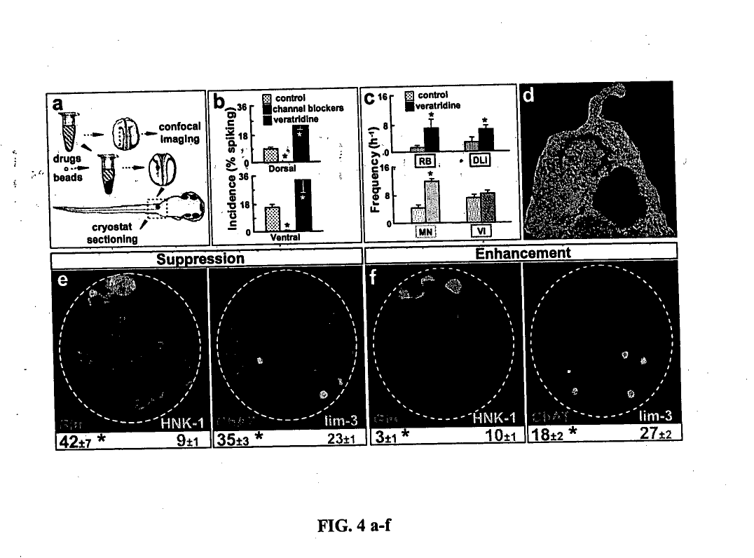

To check the results of channel expression and to focus the manipulation of Ca²⁺ spikes on the period when this activity is manifested after closure of the neural tube, a pharmacological approach was adopted. We implanted agarose beads²⁶ immediately before the time of tube closure, loaded either with Ca²⁺ spike blockers²⁷ or with veratridine to activate Na⁺ channels and increase Ca²⁺ spike activity (FIG. 4a). In FIG. 4a, Ca²⁺ imaging was performed on stage 22-26 embryos (boxed region in FIG. 4a) in the presence of pharmacological agents; single agarose beads loaded with these agents were implanted adjacent to the nascent neural tube at stage 17-18, and stage 40 larvae were sectioned for immunocytochemistry. The effectiveness of these agents was demonstrated by the suppression of spontaneous activity by the blockers and its enhancement by veratridine (FIG. 4b, c), with no effect of bovine serum albumin (BSA, control) when applied in the bath during imaging experiments. In FIGS. 4b and c, dotted columns represent controls; hatched columns, channel blockers; solid columns, veratridine. In FIGS. 4b and c, n≧10 neural tubes were analyzed. After implantation (FIG. 4d), Glu-IR and ChAT-IR were bilaterally increased when beads contained blockers, suppressed when beads contained veratridine (FIG. 4e, f), and unaffected when beads contained BSA. In FIG. 4d, the bead is black and the neural tube is outlined in with a dashed circle. For each condition, n≧5. As above, asterisks indicate significantly different from control, in 4b, c, e and f. The difference between the effects of unilateral channel overexpression and unilateral bead implantation might be due to the diffusion of agents in the latter case. The results of these pharmacological perturbations confirm and extend the results of channel overexpression and demonstrate an inverse relationship between Ca²⁺ spike activity and the expression of excitatory transmitters.

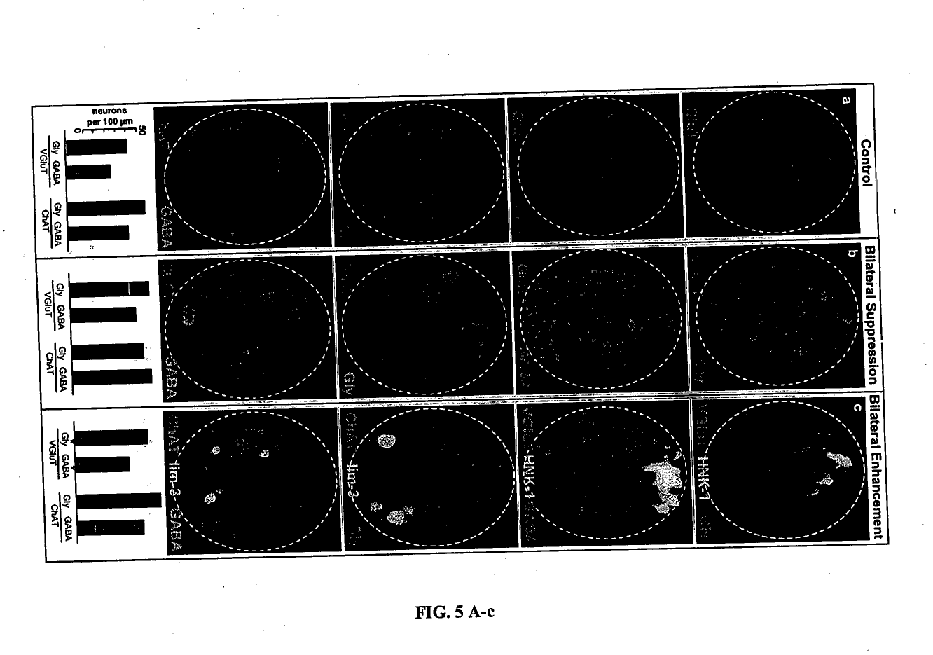

To test the idea that transmitter specification is regulated homeostatically by Ca²⁺ spike activity, we investigated the expression of GABA and glycine. These transmitters are inhibitory for most of these embryonic Xenopus spinal neurons,²⁸ although they are excitatory in other developing systems. Decreases in expression of Gly-IR and GABA-IR accompanied the increases in Glu-IR and ChAT-IR after bilateral suppression of activity (FIG. 5a, b). In FIG. 5a, controls are doubly stained for glutamate or ChAT (dark gray, excitatory) plus glycine or GABA (light gray, inhibitory) and illustrate the normal distribution of immunoreactivity. In FIG. 5b, embryos in which spike activity was bilaterally suppressed by expression of hKir2.1 are stained as in a; lighter gray indicates coexpression of excitatory and inhibitory transmitters. The increased incidence of excitatory transmitters seemed to lead to coexpression with inhibitory transmitters in some cases. In others, it was correlated with the disappearance of glycine and GABA, indicating the possible replacement of inhibitory by excitatory transmitters. In a reciprocal manner, the decrease in Glu-IR and ChAT-IR after bilateral enhancement of activity was accompanied by an increase in the incidence of GABA⁺ and glycine⁺ neurons relative to controls. Overexpression of rNaᵥ2aαβ led to the appearance of RB immunoreactive for HNK-1 and GABA or glycine, in the presence or absence of Glu-IR. Similarly, we observed MN immunoreactive for lim-3 and glycine or GABA, in the presence or absence of ChAT-IR (FIG. 5c). In FIG. 5c, embryos in which spike activity was bilaterally enhanced by expression of rNaᵥ2a are stained for glutamate or ChAT and the respective marker of cell identity (HNK-1 or lim-3, green), plus glycine or GABA. Lightest gray denotes coexpression of marker and inhibitory transmitter; white shows coexpression of marker and both excitatory and inhibitory transmitters. At the bottom of FIG. 5, the chart shows the number of neurons immunoreactive for different transmitters per 100 μm of neural tube, means ±s.e.m.; n≧5 for each condition. In FIGS. 5b and c, asterisks indicate significantly different from control. These findings imply homeostatic regulation of transmitter expression by activity, overriding other developmental cues. Down-regulation of activity seems to stimulate the production of excitatory transmitters in inhibitory neurons and to suppress the expression of inhibitory transmitters in neurons that normally express them. Up-regulation of activity leads to an increased incidence of neurons expressing inhibitory transmitters and to the expression of inhibitory transmitters in neurons normally expressing excitatory transmitters.

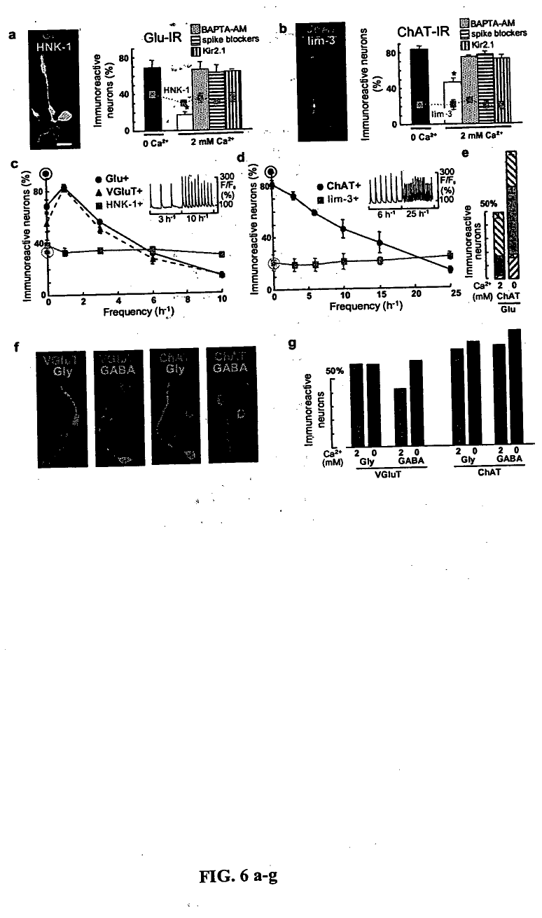

To determine more precisely the effect of different Ca²⁺ spike frequencies on neurotransmitter expression, we examined neurons in low-density-dissociated cell cultures in the absence of synaptic connections. Suppression of spike activity during development in vitro increased the incidence of expression of Glu-IR and ChAT-IR, paralleling observations in vivo, without affecting the number of neurons expressing RB and MN markers of cell identity (HNK-1 and lim-3; FIG. 6a, b). FIGS. 6a and b show data from at least five cultures containing n≧40 neurons. (Asterisks indicate significantly different from 0 mM Ca²⁺.) We then imposed various Ca²⁺ spike frequencies on differentiating neurons and examined Glu-IR, VGluT-IR and ChAT-IR. The expression of HNK-1 and lim-3 was constant across the range of frequencies of imposed Ca²⁺ spikes, whereas the incidence of Glu⁺/VGluT⁺ or ChAT⁺ neurons varied inversely with frequency. Low frequencies enhanced the expression of these transmitters, and high frequencies suppressed their expression (FIG. 6c-e). In FIG. 6c, HNK-1⁺ neurons are Glu⁺/VGluT⁺ at frequencies of 6 h⁻¹ or lower. Bull's eyes indicate the effect of stimulation with the RB pattern of spikes. (See FIG. 1f.) In the FIG. 6c inset, note the examples of simulated spikes at 3 and 10 h⁻¹. Note that in FIG. 6d, lim-3+ neurons are ChAT⁺ at frequencies of 15 h⁻¹ or lower. Bull's eyes indicate the effect of stimulation with the MN pattern of spikes. (See FIG. 1f.) In the FIG. 6d inset, note the simulated spikes at 6 and 25 h⁻¹. FIG. 6e shows proportions of Glu⁺/ChAT⁺ doubly stained neurons when cultured in 2 mM Ca²⁺ or 0 mM Ca²⁺ medium (indicated by 2 or 0, respectively). The percentage of neurons doubly labeled for glutamate and ChAT is consistent with predictions from 6a-d. The developmental progression of spike frequencies observed in the neural tube (FIG. 1f) was most effective in enhancement. These findings are consistent with results in vivo and provide further information about the spike activity patterns that are sufficient to regulate transmitter expression. However, transmitter expression also depends on factors related to cell identity over a wide range of the frequencies tested, because cells immunopositive for HNK-1 or lim-3 were preferentially immunoreactive for glutamate or ChAT. Activity overrides this dependence on intrinsic cell identity at the higher Ca²⁺ spike frequencies. The balance of excitatory and inhibitory neurotransmitters changed with the level of spike activity: low activity (0 mM Ca²⁺, spikes absent) stimulated increases in excitatory and decreases in inhibitory transmitters. In contrast, high activity (2 mM Ca²⁺, spikes present at 10 h⁻¹)²⁷ stimulated increases in inhibitory and decreases in excitatory transmitters (FIG. 6f, g). Note that FIGS. 6e and f show data from at least five cultures containing n≧40 neurons. These results are consistent with the increased incidence of expression of GABA with increasing spike frequency¹⁴ and indicate that activity-dependent regulation of transmitter expression might be cell autonomous.

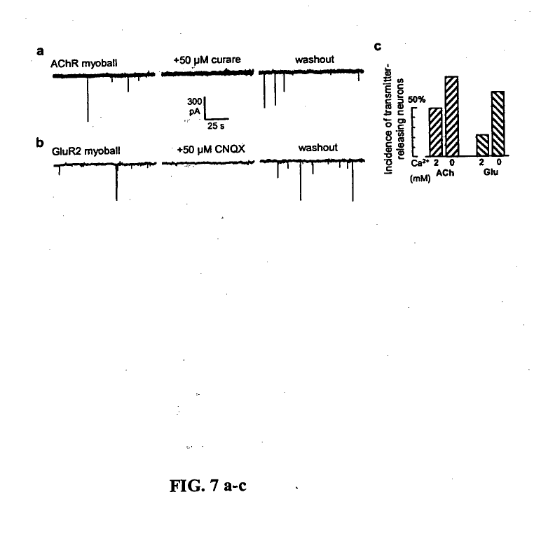

Given the changes in incidence of Glu-IR and ChAT-IR when neurons are subjected to different patterns of spike activity, we then tested whether neurons are able to release these transmitters. When acetylcholine-receptor-expressing myoballs were brought into contact with neuronal growth cones, we recorded spontaneous synaptic currents (SSCs) that were blocked by curare but not by 6-cyano-7-nitroquinoxaline-2,3-dione (CNQX). We used blastomere injection of rGluR2 messenger RNA to generate myoballs expressing glutamate receptors; when these myoballs were brought in contact with growth cones, we recorded SSCs that were blocked by CNQX but not by curare. The absence of cholinergic SSCs in this condition might have been due to down-regulation of AChR by overexpression of GluR2. The percentages of neurons generating glutamatergic and cholinergic SSCs when cultured in medium containing 0 or 2 mM Ca²⁺ (FIG. 7; n≧10) were not distinguishable from the incidence of Glu-IR and ChAT-IR (FIG. 6a, b), indicating that these altered phenotypes are functionally expressed. Note that FIG. 7, depicting whole-cell recording from a myoball expressing native AChR manipulated in front of the growth cone of a neuron grown in 2 mM Ca²⁺ culture medium, illustrates control SSCs in the absence and presence of curare. A parallel analysis of evoked synaptic currents elicited by the electrical stimulation of neurons yielded similar percentages. The results can account for the high incidence of neuromuscular junctions in nerve-muscle cocultures.²⁹

We further tested for the presence of activity-dependent homeostatic specification of neurotransmitter receptors. We found that changing the neurotransmitter in a population of neurons results in the necessary change of receptors in the postsynaptic cells to make the presynaptic neurotransmitter change functional.^{30,31}

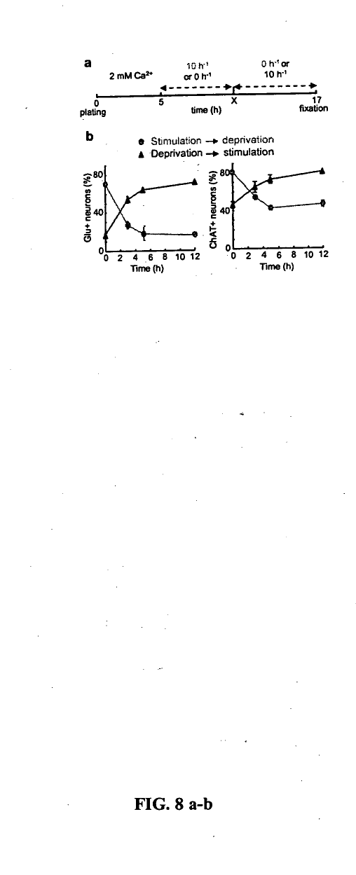

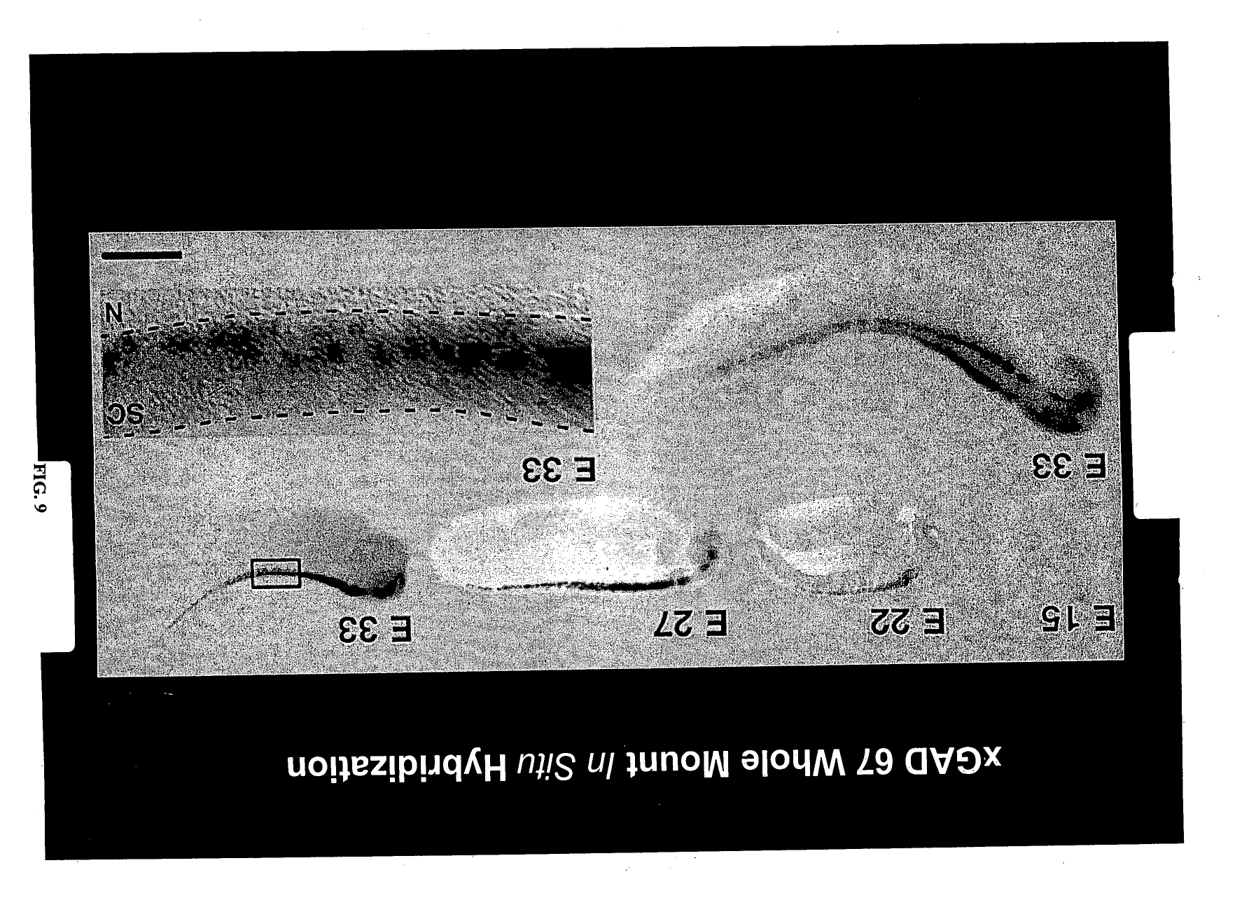

Regulatory roles of activity during neuronal development are often confined to critical periods.^{3,33} To determine whether this is true of transmitter expression, we suppressed or imposed spikes for various times in vitro to identify the minimal stimulation required to achieve changes in incidence of transmitter expression. To test the existence of critical periods, growth in 2 mM Ca²⁺ for 5 h after plating was followed by exchange of the culture medium for one containing 0 mM Ca²⁺ (deprivation) or by the imposition of spikes at 10 h⁻¹ (stimulation) for various periods. Treatment was reversed after X hours, and cultures were fixed and stained for glutamate or ChAT at 17 h in culture. Five-hour periods of perturbation beginning near the time at which Ca²⁺ spikes are first expressed are sufficient to increase or decrease the incidence of Glu-IR or ChAT-IR to the same extent as that attained after 12 h. These seem to be critical periods, because the reversal of stimulation or deprivation after 5 h is ineffective in reversing the changes during the periods tested (FIG. 8). (Note that in both parts of FIG. 8b, n≧5 cultures were used containing n≧40 neurons.) We further found that the early expression of multiple neurotransmitters is pruned to single transmitters by electrical activity, and that the early expression of transmitters regulates this electrical activity.^{34,35} FIG. 9 depicts the developmental up-regulation of xGAD 67 transcripts, which is first detected at the neural tube stage of developing Xenopus embryos. Whole mount in situ hybridization defines the spatial as well as temporal expression of xGAD 67 transcripts. Top row: mRNA is not detected in the neural plate of gastrula stage (E15) embryos, but is expressed in the spinal cord and brain at early neural tube stages (E22). A similar pattern of xGAD 67 mRNA expression is observed in stage 27 and 33 embryos; the signal has now extended caudally in the spinal cord and rostrally in the brain. Anterior is to the left in all views. E15, dorsal view; E22-33, lateral views and dorsal up. Bottom row: dorsolateral view reveals two parallel rows of labeled cells that diverge anteriorly; stage 33. Sagittal 10 μm section of a stage 33 embryo (from region indicated by box above) demonstrates label in equatorially and ventrally situated cells in the positions of ascending interneurons and Kolmer-Agduhr cells. Embryos were hybridized to either a digoxigenin xGAD 67 antisense or sense control cRNA probe. Reaction product was absent from stage-matched sense controls. Scale bar top row: 0.9, 0.9, 1.0, 1.75 mm; bottom row: 1.1 mm, 80 μm.

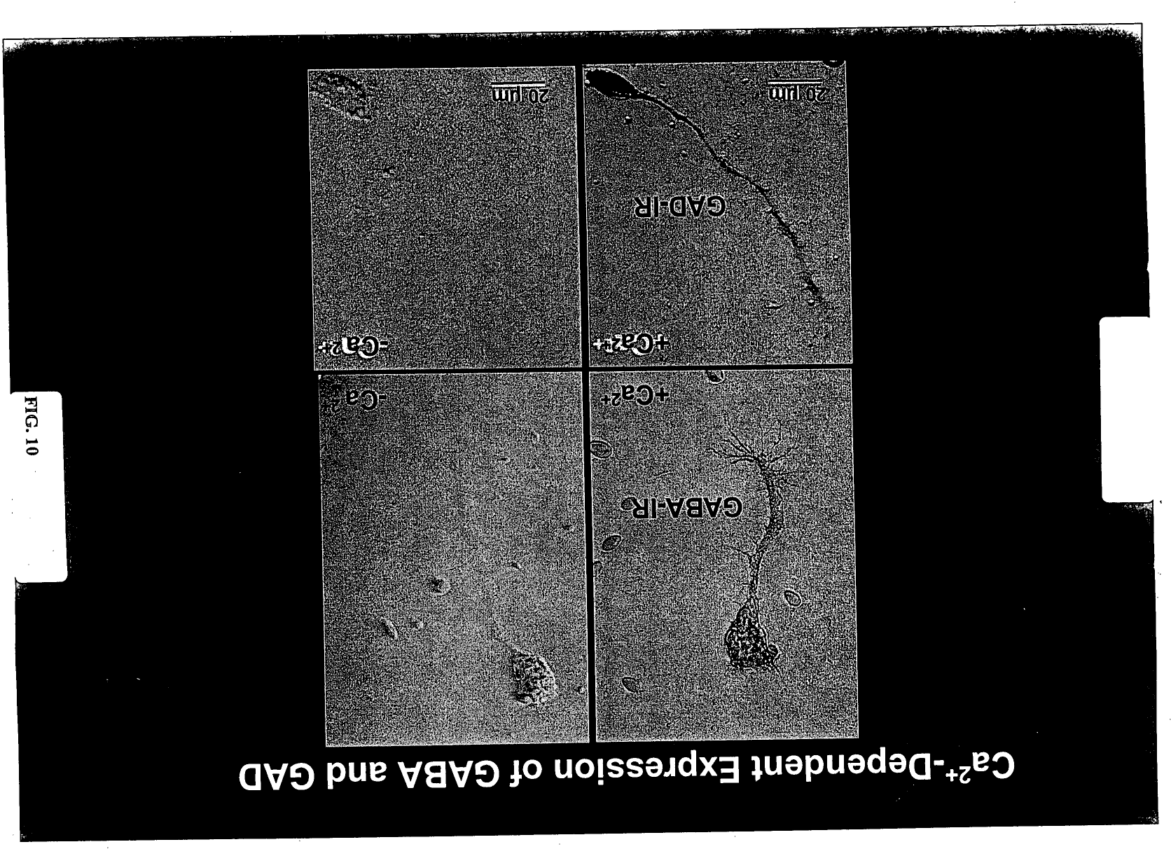

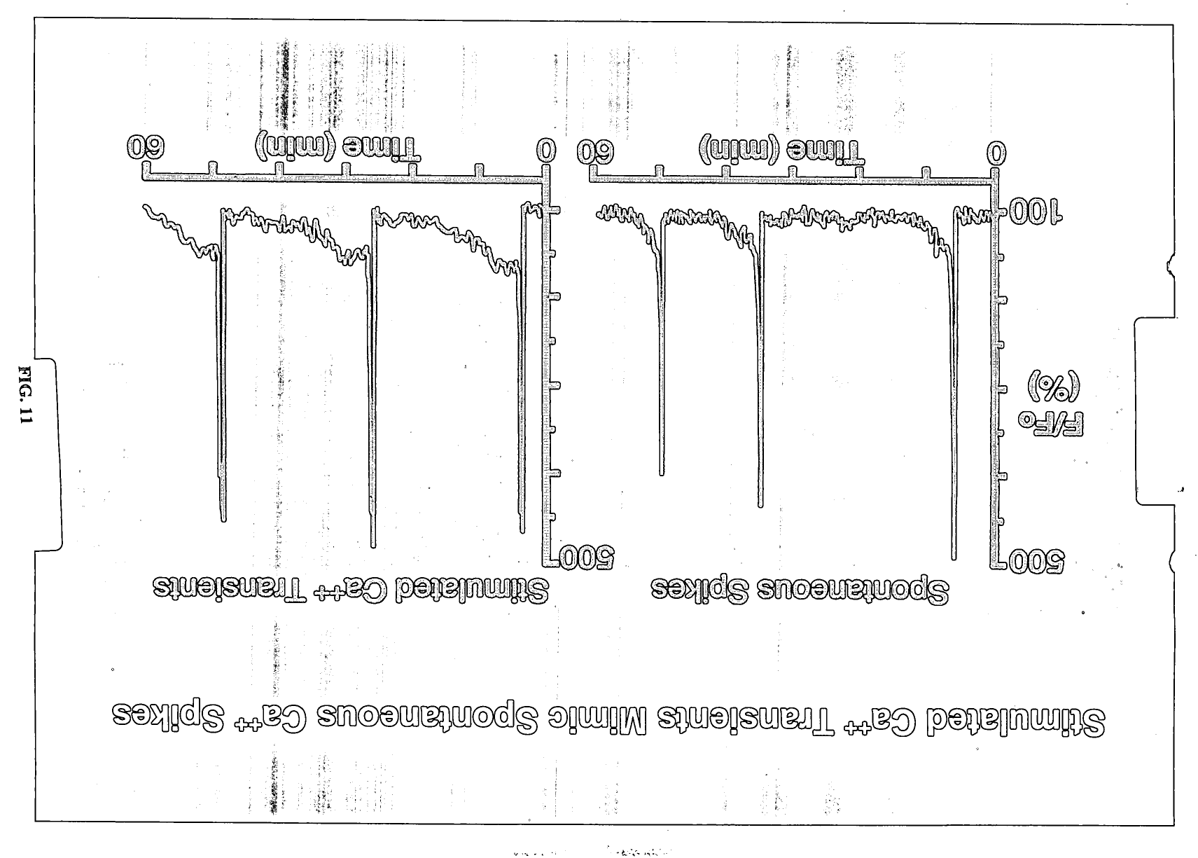

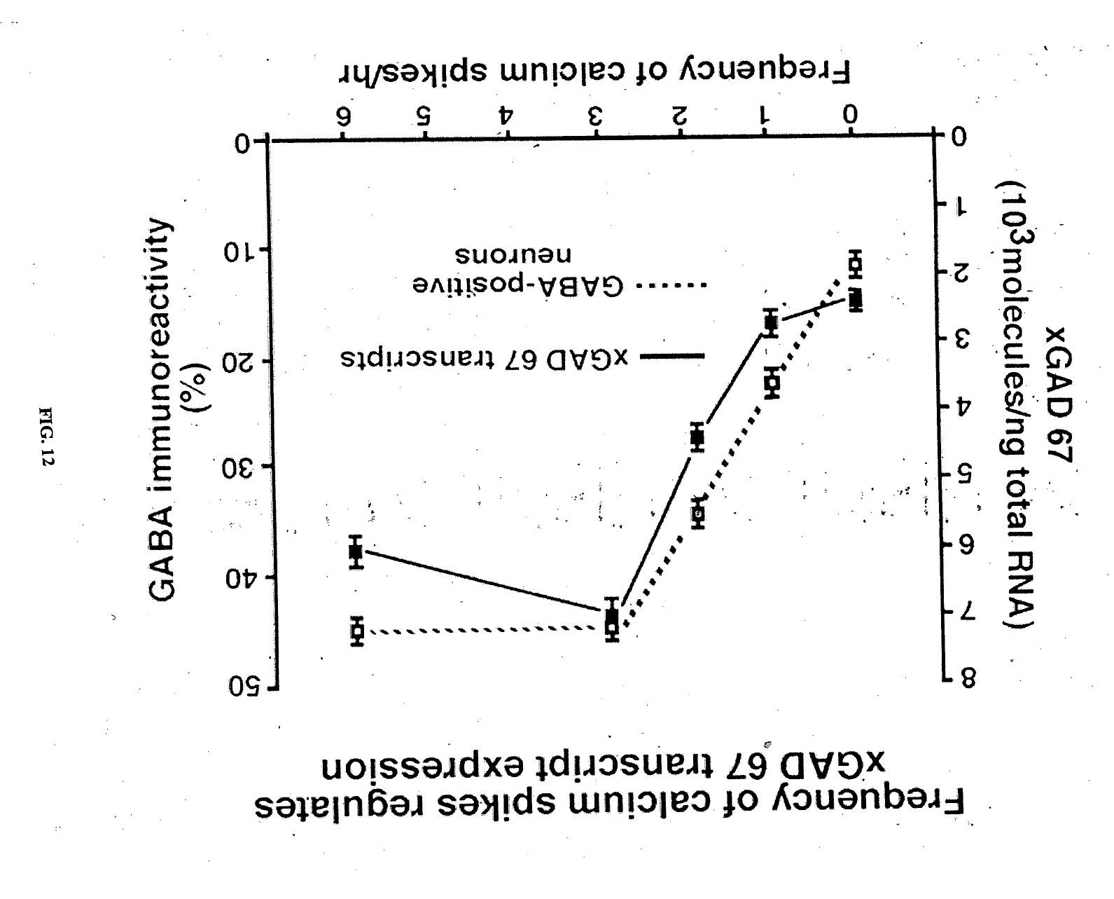

Our results indicate that specific patterns of Ca²⁺ spike activity are necessary for normal expression of neurotransmitters in neurons in the embryonic spinal cord, because disrupting these patterns alters transmitter expression. Neuronal death or neurogenesis seems unlikely to contribute to these results because the numbers of RB and MN identified by HNK-1 and lim-3 do not change in vivo or in vitro. Ca²⁺ spike activity might also be sufficient to drive the expression of particular transmitters, because patterns of stimulation that more closely parallel the in vivo activity of RB or MN generate an increasing proportion of neurons expressing glutamate or ChAT in vitro. However, a large percentage of these neurons are immunoreactive for both glutamate and ChAT, and the mechanism by which neurons are led to express a single classical transmitter during normal development in vivo remains to be determined. Candidates include regulation by transcription factors, more subtle distinctions among patterns of spontaneous activity, the presence of synaptic activity, and signal transduction by means of protein factors. Natural changes in spike activity patterns could have a role in the restriction of the extensive early expression of GAD and GABA.³⁶ As FIG. 10 demonstrates, GABA neurotransmitter and GAD enzyme expression in neurons depends on the presence of extracellular calcium. Immunocytochemical analysis illustrates the presence of GABA (top left) and GAD (bottom left) throughout neurons cultured in the presence of calcium; GABA and GAD immunoreactivity are absent from neurons grown in the absence of calcium (bottom left and right). Furthermore, as FIG. 11 depicts, spontaneous spike patterns can be mimicked by stimulation of neurons in culture. Elevations of intracellular calcium in a neuron are generated by application of 20-30 second pulses of high concentrations of potassium chloride with calcium chloride at 3/hr, which mimic spontaneous calcium spikes. F/F₀ indicates fluorescence increase above baseline; 8 h in culture. And, as FIG. 12 demonstrates, competitive Quantitative Reverse Transcription-Polymerase Chain Reaction (QRT-PCR) analysis of xGAD 67 expression in neurons grown ±calcium+ or following different frequencies of experimentally imposed calcium transients. Numbers of transcripts increase from 2400±0/ng to 7000±200/ng total RNA (2.9±0.1-fold) as the frequency increases in the physiological range from 0-3/hr. The increase in transcript number parallels the percent of GABA-immunoreactive neurons.¹³ N=5 experiments for each condition. The co-expression of several transmitters in single embryonic neurons after particular patterns of activity might provide the basis for transmitter co-expression in mature neurons.³⁷

Shifts in transmitter expression in response to decreases or increases in spike activity seem to be directed towards the homeostasis of network excitability in the nervous system.³⁸ This process is conceptually similar to the homeostatic resetting of neuronal excitability and synaptic strengths that provides an important balance to hebbian plasticity^{39,40} and to the homeostatic plasticity of excitatory networks that renders spontaneous output resistant to disruption of connectivity.^{41,42} It was thus surprising to observe this process in neurons in dissociated cell culture in the absence of synaptic connections, in turn implying that the feedback loop might be intracellular. This regulatory program indicates that abnormal electrical activity could regulate neurotransmitter expression. Although high frequencies of Ca²⁺ spikes generated in pediatric epilepsy can be detrimental to brain development, seizure activity seems to regulate the expression of neurotransmitter homeostatically to suppress this activity. Mossy fibers normally generate excitatory glutamatergic postsynaptic potentials on hippocampal CA3 pyramidal cells, but generate inhibitory GABAergic postsynaptic potentials after kindled seizures.⁴³

The effects of activity on transmitter specification probably operate through dynamic interplay with the molecular context provided by transcription factors. We propose a model in which the hierarchical expression of transcription factors defines fields of cells⁴⁴⁻⁴⁶ that express constellations of ion channels. These channels produce patterns of activity that are modulated by protein factors. This modulated activity further engages transcription factors that stipulate transmitter expression. The transmitter that is specified then depends on the transcription factors expressed by the postmitotic neuron, the appropriate type of activity, interaction with signalling proteins, and further transcriptional regulation. This scheme enables the integration of genetic coding with signals that stimulate spike activity or the secretion of factors. A consequence of this proposal is that knockouts of neuronal class-specific transcription factors might not lead to transmitter switches unless they are involved in programming electrical activity.⁴⁷ Moreover, the robustness of transmitter phenotype in cells dissociated and grown in culture⁴⁸ implies the preservation of patterns of activity as observed in this study. Consistent with this model is the observation that misexpression of a lim-class homeobox gene in epidermal cells leads to ectopic expression of a putative voltage-dependent Na⁺ channel gene normally expressed in motor neurons,⁴⁹ and neurotrophins modulate Ca²⁺ and K⁺ channels.⁵⁰ The signal transduction cascades by which these low frequencies of spikes exert these effects are likely to engage RNA synthesis^{14,45} by means of activity-dependent transcription factors.^{51,52}

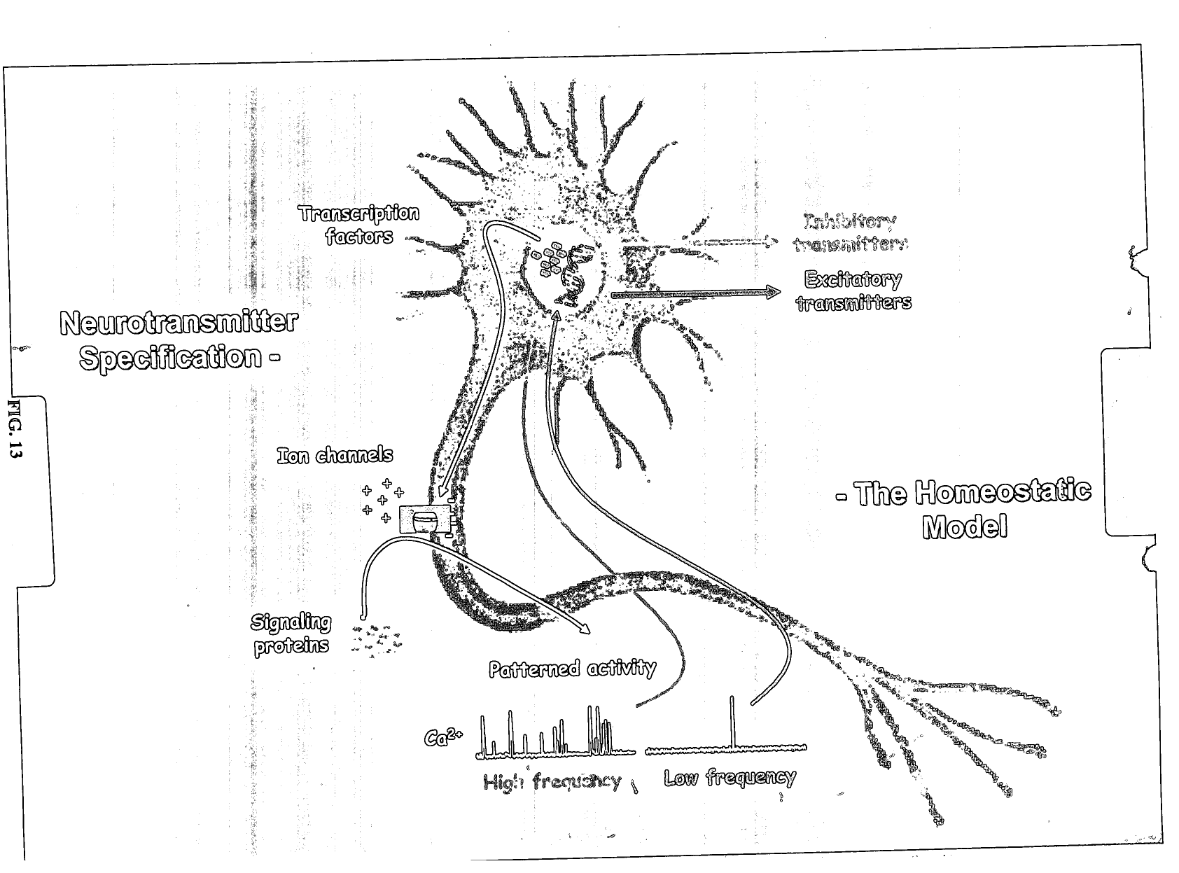

Thus, on the one hand, activity-dependent changes in transmitter expression can be expected to affect axon guidance. Growth cones of developing Xenopus neurons release transmitter spontaneously⁵³ and turn in response to acetylcholine, glutamate and GABA.^{54,55} In agreement with this view is the finding that the depletion of transmitters in vivo alters axon outgrowth.⁵⁶ On the other hand, neurons rerouted by novel neurotransmitter expression can be expected to make synaptic connections with novel postsynaptic partners. Embryonic Xenopus neurons express multiple classes of transmitter receptors,²⁸ providing key components necessary for the formation of functional connections. Alternatively, the expression of postsynaptic receptors might be regulated homeostatically in parallel with the regulation of transmitter expression. However, mismatches between presynaptically released transmitter and postsynaptically expressed receptors may arise. FIG. 13 summarizes the homeostatic model, which forms the basis of our view of neurotransmitter specification. To summarize, expression of transcription factors identifies classes of neurons that express constellations of ion channels. These channels produce patterned Ca²⁺ spike activity that is modulated by signaling proteins. Patterns of spike activity, activating Ca²⁺-dependent transcription factors, regulate expression of transcripts encoding the enzymes that synthesize and store specific transmitters. Different levels of activity homeostatically specify expression of excitatory and inhibitory transmitters.

### EXAMPLES

The present invention can be used in conjunction with magnetoencephalography (MEG). MEG is the measurement of magnetic fields generated by electric currents in the brain. Measurement of these fields close to the surface of the head allows localization of the origin of the electric currents and may be used to map cortical brain function. MEG provides millisecond temporal resolution and millimeter spatial resolution of brain function, but no detailed anatomical information. It is therefore often combined with MR imaging, the merged data set being named magnetic source imaging MSI.

MEG is based on the principle that all electric currents generate magnetic fields. The main source of the extracranial magnetic fields that are detected with MEG instruments is current flow in the long apical dendrites of the cortical pyramidal cells. A distal excitatory synapse will induce a dipolar dendritic current towards the soma of the pyramidal cell, meaning that the electricity is flowing in one direction along the entire length of the dendrite, which therefore may be considered an electric dipole. Pyramidal neurons constitute nearly 70% of neocortical neurons, and the cells are oriented with their long apical dendrites perpendicular to the brain cortex. There are more than 100,000 of these cells per square millimeter of cortex. Dipolar currents flowing in these dendrites induce time-varying magnetic fields perpendicular to the dendrite direction. The pattern of these external magnetic fields can be used to determine the location, orientation and strength of the source electric dipoles.

Neuromagnetic fields have amplitudes in the order of a few picotesla (10-12 tesla) and very sensitive instruments are needed to detect these extremely weak fields. Most MEG instruments (magnetometers, biomagnetometers) are placed in special magnetically shielded rooms. The walls have one or more layers of mu metal, an alloy with very high magnetic permeability, mounted on an aluminum plate serving as magnetic and electromagnetic shielding. External magnetic fields follow the mu metal around the room, away from the interior MEG instrument. MEG detection systems are made from superconductive material immersed in liquid helium. MEG detectors are specially designed coils, the most common one being named axial first-order gradiometer. This coil consists of two coil loops wound in opposite directions, typically less than 4 cm apart. The time-varying external neuromagnetic fields induce electric currents in both loops, the strength of the currents being determined by the strength of the magnetic fields. If the loop currents had identical strengths, they would cancel and no signal would emanate from the detector. Dipolar magnetic fields diminish with the square of the distance from the dipolar source and the loop closest to the brain will therefore experience a slightly stronger field than the loop more distant from the brain. The net output from the detection coil is thus proportional to the magnetic field gradient. The gradient is steep close to the source and shallow far from the source. This makes the detector more sensitive to a very close weak source (such as the brain a few centimeters away), than to a strong, very distant source (such as an MR scanner some hundred feet away).

The detection coil is inductively coupled to a SQUID (superconducting quantum interference device). This is a ring of superconducting material interrupted by two microscopically thin resistive segments (Josephson junctions). A small current is applied to the ring, and provided the current is below a certain critical value, the current will flow without resistance despite the two tiny resistive segments. Any increase in the SQUID current above the critical value will cause a significant drop in the current due to sudden energy loss in the resistive segments. The SQUID current is kept just below the critical value and any induced additional current caused by a net output from the detection coil, will cause a significant drop in the SQUID voltage. The voltage drop is detected by the electronics, which applies a feedback current to counterbalance the induced current in the SQUID. The output from the MEG instrument is determined by the magnitude of the feedback current as measured by a voltmeter. Modern MEG instruments have multiple (e.g., 37) detectors, so-called large-array biomagnetometers. Whole-head systems may have dual multi-channel detectors for simultaneous bilateral recordings, or the dewar containing the multiple detectors may have a helmet-like shape. Large, flat detector systems intended for measurement of biomagnetic fields from the heart, also exist.

The recorded biomagnetic signals are very similar to EEG, and also similar to EEG the signals may be either spontaneous or related to some stimulus (audiovisual, tactile, vibratory, electric, etc.). MEG may be used to explore normal brain function, to map brain function in the vicinity of a tumor or epileptic focus prior to surgery or radiation therapy, to image epileptic foci, to monitor recovery after stroke or head trauma and to study the effects of neuropharmacological agents.

Imaging: Neural tubes dissected from three embryonic epochs²⁰ and dissociated cell cultures prepared from neural-plate-stage embryos³³ were loaded with 5 μM fluo-4 acetoxymethyl ester or 1 mM bisoxonol, and images were acquired at 0.2 Hz for 1-h periods with a BioRad MRC1024 laser confocal system. Spikes were stimulated at different frequencies in vitro by culturing neurons in 250 ml Ca²⁺-free saline medium by using a volume reducer and continuously superfusing them with this medium at 2.5 ml min⁻¹. The composition of saline was automatically switched for 15-20 s by computer-controlled solenoid valves (General Valve Corp.) to a solution containing 100 mM KCl and 2 mM Ca²⁺.

Molecular biology and pharmacology: hKir2.1, rNav2aab and rGluR2 were gifts from E. Marban, W. Catterall, and S. Heinemann. The genes were subcloned into a Bluescript vector and complementary DNA was transcribed with the mMessage mMachine (Ambion). Capped RNA (5-10 nl of a 0.01-0.1 mg ml⁻¹ RNA solution in 10% MMR, 6% Ficoll) was co-injected with a Cascade blue or Rhodamine red 30-kDa dextran (30 mg ml⁻¹) into one or both blastomeres at the two-cell stage with the use of a picospritzer (Picospritzer III; Parker Instrumentation). Control injections consisted of fluorescent dextran alone. Agarose beads (80 μm; BioRad) were loaded for 1 h with a solution containing 200 nM calcicludine (Calbiochem), 10 μM GVIAq-conotoxin, 10 μM flunarizine and 10 μm tetrodotoxin, or 1 mM veratridine, or 1% BSA (all from Sigma) before implantation. The effect of most of these agents on spike activity was tested at one-tenth of these concentrations; veratridine was tested at one-thousandth.

Immunocytochemistry: Embryos were fixed in 4% paraformaldehyde, 0.1% glutaraldehyde, phosphate-buffered saline (pH 7.4) for 2 h at 4° C., soaked in 30% sucrose for 2.5 h, and embedded in OCT compound (Tissue-Tek, Fisher Scientific). Frozen sections 10 μm in thickness were made over a 400-μm region of the neural tube starting about 400 μm posterior to the back of the eyes for control and channel-misexpression embryos. In bead-implanted embryos the bead was inserted between the neural tube and myotomes about 400 μm behind the eye primordium and sectioned from 100 μm anterior to 100 μm posterior to the bead. Cultures were fixed for 5-10 min with the same fixative. Slides and cultures were incubated in a blocking solution of 1% goat serum, fish gelatin or BSA for 0.5 h at 20° C., followed by incubation overnight at 4° C. with primary antibodies, and incubation for 2 h with fluorescently tagged secondary antibodies at 20° C. Immunoreactivity was examined on a Zeiss Photoscope with a 40× oil-immersion objective (or 20× and 40× water-immersion objectives for cultures) using a Xenon arc lamp attenuated by neutral density filters, and the appropriate excitation and emission filters for Cascade Blue, Alexa 488 and Alexa 594 fluorophores. Images were acquired and analyzed with Metamorph software (Universal Imaging Corp.). Each phenotype was scored in 20-30 consecutive sections or in cultures from at least five embryos. Immunoreactivities are presented in saturated color to clarify the distinction between positive and negative cells and to normalize image intensities. Differences from control were considered significant at p<0.05 (Student's t-test). Antibodies were from Affinity Bioreagents, Calbiochem, Chemicon and Sigma.

Electrophysiology: Cultured neurons and myocytes (myoballs) were observed with an upright compound microscope and a 40× water-immersion objective that allowed the positioning of electrodes with phase-contrast optics. Whole-cell recording¹³ was used to record SSCs. A Dell Dimension 4100 computer with Axon Instruments (PClamp 8.1) software and data interfaces was used for the acquisition and analysis of currents. Electrodes were pulled from borosilicate capillaries and had resistances of 3-5MΩ; they were filled with 100 mM KCl, 10 mM EGTA, 10 mM HEPES, pH 7.4. A perfusion system allowing rapid change of the bathing medium was used to achieve solution changes (2 ml min⁻¹). The receptor dependence of currents was determined by adding various drugs.
1. Snyder, S. H. and Ferris, C. D. Novel neurotransmitters and their neuropsychiatric relevance. Am. J. Psychiatry 157, 1738-1751 (2000).
2. Zafra, F., Hengerer, B., Leibrock, J., Thoenen, H., and Lindholm, D. Activity dependent regulation of BDNF and NGF mRNAs in the rat hippocampus is mediated by non-NMDA glutamate receptors. EMBO J. 9, 3545-3550 (1990).
3. Tanabe, Y., William, C., and Jessell, T. M. Specification of motor neuron identity by the MNR2 homeodomain protein. Cell 95, 67-80 (1998).
4. Thaler, J. P., Lee, S. K., Jurata, L. W., Gill, G. N., and Pfaff, S. L. LIM factor Lhx3 contributes to the specification of motor neuron and interneuron identity through cell-type-specific protein-protein interactions. Cell 110, 237-249 (2002).
5. Pierani, A. et al. Control of interneuron fate in the developing spinal cord by the progenitor homeodomain protein Dbx1. Neuron 29, 367-384 (2001).
6. Jin, Y., Hoskins, R., and Horvitz, H. R. Control of type-D GABAergic neuron differentiation by C. elegans UNC-30 homeodomain protein. Nature 372, 780-783 (1994).
7. Thor, S. and Thomas, J. B. The Drosophila islet gene governs axon pathfinding and neurotransmitter identity. Neuron 18, 397-409 (1997).
8. Furshpan, E. J., MacLeish, P. R., O'Lague, P. H., and Potter, D. D. Chemical transmission between rat sympathetic neurons and cardiac myocytes developing in microcultures: evidence for cholinergic, adrenergic, and dual-function neurons. Proc. Natl. Acad. Sci. USA 73, 4225-4229 (1976).
9. Landis, S. C. and Keefe, D. Evidence for neurotransmitter plasticity in vivo: developmental changes properties of cholinergic sympathetic neurons. Dev. Biol. 98, 349-372 (1983).
10. Nawa, H. and Patterson, P. H. Separation and partial characterization of neuropeptide-inducing factors in heart cell conditioned medium. Neuron 4, 269-277 (1990).
11. Walicke, P. A. and Patterson, P. H. On the role of Ca²⁺ in the transmitter choice made by cultured sympathetic neurons. J. Neurosci. 1, 343-350 (1981).
12. Rao, M. S., Tyrrell, S., Landis, S. C., and Patterson, P. H. Effects of ciliary neurotrophic factor (CNTF) and depolarization on neuropeptide expression in cultured sympathetic neurons. Dev. Biol. 150, 281-293 (1992).
13. Gu, X. and Spitzer, N. C. Distinct aspects of neuronal differentiation encoded by frequency of spontaneous Ca²⁺ transients. Nature 375, 784-787 (1995).
14. Watt, S. D., Gu, X., Smith, R. D., and Spitzer, N. C. Specific frequencies of spontaneous Ca²⁺ transients up-regulate GAD 67 transcripts in embryonic spinal neurons. Mol. Cell. Neurosci. 16, 376-387 (2000).
15. Hartenstein, V. Early pattern of neuronal differentiation in the Xenopus embryonic brainstem and spinal cord. J. Comp. Neurol. 328, 213-231 (1993).
16. Li, W. C. et al. Defining classes of spinal interneuron and their axonal projections in hatchling Xenopus laevis tadpoles. J. Comp. Neurol. 441, 248-265 (2001).
17. Hanson, M. G. and Landmesser, L. T. Characterization of the circuits that generate spontaneous episodes of activity in the early embryonic mouse spinal cord. J. Neurosci. 23, 587-600 (2003).
18. Gu, X., Olson, E. C., and Spitzer, N. C. Spontaneous neuronal calcium spikes and waves during early differentiation. J. Neurosci. 14, 6325-6335 (1994).
19. Somasekhar, T. and Nordlander, R. H. Selective early innervation of a subset of epidermal cells in Xenopus may be mediated by chondroitin sulfate proteoglycans. Dev. Brain Res. 99, 208-215 (1997).
20. Arber, S. et al. Requirement for the homeobox gene Hb9 in the consolidation of motor neuron identity. Neuron 23, 659-674 (1999).
21. Bernhardt, R. R., Patel, C. K., Wilson, S. W., and Kuwada, J. Y. Axonal trajectories and distribution of GABAergic spinal neurons in wildtype and mutant zebrafish lacking floor plate cells. J. Comp. Neurol. 326, 263-272 (1992).
22. Appel, B. et al. Motoneuron fate specification revealed by patterned LIM homeobox gene expression in embryonic zebrafish. Development 121, 4117-4125 (1995).
23. Taira, M., Hayes, W. P., Otani, H., and Dawid, I. B. Expression of LIM class homeobox gene Xlim-3 in Xenopus development is limited to neural and neuroendocrine tissues. Dev. Biol. 159, 245-256 (1993).
24. Schneider, C., Wicht, H., Enderich, J., Wegner, M., and Rohrer, H. Bone morphogenetic proteins are required in vivo for the generation of sympathetic neurons. Neuron 24, 861-867 (1999).
25. Gorbunova, Y. V. and Spitzer, N. C. Dynamic interactions of cyclic AMP transients and spontaneous Ca²⁺ spikes. Nature 418, 93-96 (2002).
26. Bixby, J. L. and Spitzer, N. C. The appearance and development of neurotransmitter sensitivity in Xenopus embryonic spinal neurons in vitro. J. Physiol. (Lond.) 353, 143-155 (1984).
27. Henderson, L. P., Smith, M. A., and Spitzer, N. C. The absence of calcium blocks impulse-evoked release of acetylcholine but not de novo formation of functional neuromuscular synaptic contacts in culture. J. Neurosci. 4, 3140-3150 (1984).
28. Borodinsky, L. N. and Spitzer, N. C. The neurotransmitter sensitivity of myocytes is regulated by calcium spike activity in embryonic spinal neurons. Society for Neuroscience 2004 Abstract (submitted).
29. Borodinsky, L. N. and Spitzer, N. C. Activity-dependent expression of novel neurotransmitter and receptor classes at the neuromuscular junction. Society for Neuroscience 2005 Abstract (submitted).
30. Hubel, D. H. and Wiesel, T. N. The period of susceptibility to the physiological effects of unilateral eye closure in kittens. J. Physiol. (Lond.) 206, 419-436 (1970).
31. Ribera, A. B. and Spitzer, N. C. A critical period of transcription required for differentiation of the action potential of spinal neurons. Neuron 2, 1055-1062 (1989).
32. Root, C. M., Minakova, E., and Spitzer, N. C. A role for early expressed GABA in activity-dependent modulation of neurotransmitter phenotype. Society for Neuroscience 2005 Abstract (submitted).
33. Root C. M. and Spitzer, N. C. A sensitive period for activity-dependent neurotransmitter specification in the embryonic spinal cord. Society for Neuroscience 2004 Abstract (submitted).
34. Somogyi, R., Wen, X., Ma, W., and Barker, J. L. Developmental kinetics of GAD family mRNAs parallel neurogenesis in the rat spinal cord. J. Neurosci. 15, 2575-2591 (1995).
35. Jonas, P., Bischofberger, J. and Sandkuhler, J. Corelease of two fast neurotransmitters at a central synapse. Science 281, 419-424 (1998).
36. Berridge, M. J., Bootman, M. D., and Roderick, H. L. Calcium signalling: dynamics, homeostasis and remodelling. Nature Rev. Mol. Cell. Biol. 4, 517-529 (2003).
37. Turrigiano, G. G. and Nelson, S. B. Hebb and homeostasis in neuronal plasticity. Curr. Opin. Neurobiol. 10, 358-364 (2000).
38. Marek, K. W. et al. A genetic analysis of synaptic development: pre- and postsynaptic dCBP control transmitter release at the Drosophila NMJ. Neuron 25, 537-547 (2000).
39. Chub, N. and O'Donovan, M. J. Blockade and recovery of spontaneous rhythmic activity after application of neurotransmitter antagonists to spinal networks of the chick embryo. J. Neurosci. 18, 294-306 (1998).
40. Milner, L. D. and Landmesser, L. T. Cholinergic and GABAergic inputs drive patterned spontaneous motoneuron activity before target contact. J. Neurosci. 19, 3007-3022 (1999).
41. Gutierrez, R. et al. Plasticity of the GABAergic phenotype of the ‘glutamatergic’ granule cells of the rat dentate gyrus. J. Neurosci. 23, 5594-5598 (2003).
42. Briscoe, J., Pierani, A., Jessell, T. M. and Ericson, J. A homeodomain protein code specifies progenitor cell identity and neuronal fate in the ventral neural tube. Cell 101, 435-445 (2000).
43. Brosenitsch, T. A. and Katz, D. M. Expression of Phox2 transcription factors and induction of the dopaminergic phenotype in primary sensory neurons. Mol. Cell. Neurosci. 20, 447-457 (2002).
44. Groves, A. K. et al. Differential regulation of transcription factor gene expression and phenotypic markers in developing sympathetic neurons. Development 121, 887-901 (1995).
45. Thaler, J. et al. Active suppression of interneuron programs within developing motor neurons revealed by analysis of homeodomain factor HB9. Neuron 23, 675-687 (1999).
46. Wichterle, H., Lieberam, I., Porter, J. A., and Jessell, T. M. Directed differentiation of embryonic stem cells into motor neurons. Cell 110, 385-397 (2002).
47. Okada, T., Katsuyama, Y., Ono, F., and Okamura, Y. The development of three identified motor neurons in the larva of an ascidian, Halocynthia roretzi. Dev. Biol. 244, 278-292 (2002).
48. DeFazio, R. A., Pong, K., Knusel, B., and Walsh, J. P. Neurotrophin-4/5 promotes dendritic outgrowth and calcium currents in cultured mesencephalic dopamine neurons. Neuroscience 99, 297-304 (2000).
49. Li, W., Llopis, J., Whitney, M., Zlokarnik, G., and Tsien, R. Y. Cell-permeant caged InsP3 ester shows that Ca²⁺ spike frequency can optimize gene expression. Nature 392, 936-941 (1998).
50. West, A. E., Griffith, E. C., and Greenberg, M. E. Regulation of transcription factors by neuronal activity. Nature Rev. Neurosci. 3, 921-931 (2002).
51. Young, S. H. and Poo, M. M. Spontaneous release of transmitter from growth cones of embryonic neurones. Nature 305, 634-637 (1983).
52. Zheng, J. Q., Felder, M., Connor, J. A., and Poo, M. M. Turning of nerve growth cones induced by neurotransmitters. Nature 368, 140-144 (1994).
53. Xiang, Y. et al. Nerve growth cone guidance mediated by G protein-coupled receptors. Nature Neurosci. 5, 843-848 (2002).
54. Diefenbach, T. J., Sloley, B. D., and Goldberg, J. I. Neurite branch development of an identified serotonergic neuron from embryonic Helisoma: evidence for autoregulation by serotonin. Dev. Biol. 167, 282-293 (1995).
55. Demarque, M., House, D. R. C., and Spitzer, N. C. Activity-dependent regulation of the incidence of serotonergic neurons in the embryonic brain. Society for Neuroscience 2005 Abstract (submitted).
56. Dulcis, D., Khalil, A., and Spitzer, N. C. Activity-dependent specification of dopaminergic neurons in the embryonic brain. Society for Neuroscience 2005 Abstract (submitted).

1. A method of modulating a neurotransmitter composition in a neuron associated with the central nervous system, the method comprising contacting the neuron with a stimulatory or inhibitory factor that alters the pattern of Ca²⁺ spike activity of the neuron.

2. The method of claim 1, wherein the neuron is an embryonic neuron.

3. The method of claim 1, wherein the neuron is a mature neuron.

4. The method of claim 1, wherein the stimulatory factor is electrical.

5. The method of claim 1, wherein the stimulatory factor is chemical.

6. The method of claim 1, wherein the neurotransmitter composition is acetylcholine, nitric oxide, histamine, norepinephrine, a bioactive amine, an amino acid or a neuropeptide.

7. The method of claim 6, wherein the bioactive amine is selected from the group consisting of dopamine, epinephrine, norepinephrine, and serotonin.

8. The method of claim 6, wherein the amino acid is selected from the group consisting of glutamate, glycine and gamma-aminobutyric acid (GABA).

9. The method of claim 6, wherein the neuropeptide is selected from the group consisting of enkephalins, dynorphins and substance P.

10. The method of claim 1, wherein the modulation of neurotransmitter composition comprises altering neurotransmitter expression.

11. A method of treating or inhibiting a psychological disorder in a subject, the method comprising contacting the subject a stimulatory or inhibitory factor that alters the pattern of Ca²⁺ spike activity of neurons, wherein the altering of Ca²⁺ spike activity results in a modification of a neurotransmitter produced by the neurons.

12. The method of claim 11, wherein the subject is a mammal.

13. The method of claim 12, wherein the mammal is a human.

14. The method of claim 11, wherein the psychological disorder is selected from the group consisting of addiction, substance abuse, autism, dyslexia, obsessive-compulsive disorder, generalized anxiety disorder, post-traumatic stress disorder, panic attacks, social phobia, major depression, bipolar disorder and schizophrenia.

15. A method of altering neurotransmitter expression, the method comprising contacting a neuron comprising a nucleic acid sequence encoding a neurotransmitter, or a nucleic acid sequence encoding an enzyme necessary for the biosynthesis of the neurotransmitter, with a stimulatory factor that alters the pattern of Ca²⁺ spike activity of the neuron.

16. A method of screening for factors that alter neurotransmitter expression in vivo comprising contacting cultures of neurons prepared from developing embryos, loaded with a calcium indicator, with a test stimulatory or inhibitory factor, wherein the neurons are exposed to different levels of the stimulatory or inhibitory factor and time-lapse imaging is used to assess changes in the firing pattern of calcium spikes produced by the neurons.

17. The method of claim 16 wherein the stimulatory or inhibitory factor is a chemical.

18. The method of claim 16 wherein the stimulant or inhibitory factor is electrical.

19. A method of treating neurological, psychological, or psychiatric disorders, the method comprising applying one or more stimulant or inhibitory factors to one or more neurons, wherein the one or more stimulant or inhibitory factors alter the Ca²⁺ spike activity of the neuron thereby changing their expression or production of specific neurotransmitters.

20. The method of claim 19, wherein the stimulant or inhibitory factor is a chemical.

21. The method of claim 19, wherein the stimulant or inhibitory factor is electrical.

22. The method of claim 21, wherein the electrical factor is applied in a manner mimicking natural patterns of activity occurring in the brain.

23. The method of claim 19, wherein the psychological disorder is selected from the group consisting of addiction, substance abuse, autism, dyslexia, obsessive-compulsive disorder, generalized anxiety disorder, post-traumatic stress disorder, panic attacks, social phobia, major depression, bipolar disorder and schizophrenia.

24. The method of claim 19, wherein the neurological disorder is selected from the group consisting of movement disorder, tardive dyskinesia, Huntington's disease, and Parkinson's disease.

25-28. (canceled)

29. The method of claim, wherein the stimulatory factor is a chemical such as veratridine.

30. The method of claim, wherein the inhibitory factor is selected from the group consisting of curare, tetrodotoxin, flunarizine, calcicludine, and omega-conotoxin.

31-35. (canceled)
